(19) United States
(12) Patent Application Publication
(10) Pub. No.: US 2017/0020612 A1
(43) Pub. Date: Jan. 26, 2017

(54) ENDOSCOPE APPARATUS

(71) Applicant: OLYMPUS CORPORATION, Tokyo (JP)

(72) Inventor: Toru KUBOI, Hachioji-shi (JP)

(73) Assignee: OLYMPUS CORPORATION, Tokyo (JP)

(21) Appl. No.: 15/284,619

(22) Filed: Oct. 4, 2016

Related U.S. Application Data
(63) Continuation of application No. PCT/JP2015/061571, filed on Apr. 15, 2015.

(30) Foreign Application Priority Data
Apr. 22, 2014 (JP) 2014-088526

(51) Int. Cl. A61B 34/20; G02B 23/26; A61B 1/00

(52) U.S. Cl. CPC A61B 34/20; A61B 1/00006; A61B 1/00009; A61B 1/00045; G02B 23/26; A61B 2034/2061

(57) ABSTRACT

An endoscope apparatus includes an endoscope including a flexible insertion tube and a curved-shape detection sensor. The sensor includes an optical fiber that transmits detection light and a sensing part provided in at least a part of the optical fiber, and detects a curved shape of the insertion tube based on a change in characteristics of the detection light passed through the sensing part in accordance with a change in the curved shape of the optical fiber when the optical fiber curves. Apart of the optical fiber or a part of a guide member through which the optical fiber is passed is held to a component having greater torsion stiffness than any other component constituting the insertion tube.

### CROSS-REFERENCE TO RELATED APPLICATIONS

This application is a Continuation Application of PCT Application No. PCT/JP2015/061571, filed Apr. 15, 2015 and based upon and claiming the benefit of priority from prior the Japanese Patent Application No. 2014-088526, filed Apr. 22, 2014, the entire contents of all of which are incorporated herein by references.

### BACKGROUND OF THE INVENTION

1. Field of the Invention

The present invention relates to an endoscope apparatus comprising a curved-shape detection sensor that detects a curved shape of a distal insertion tube of an endoscope.

2. Description of the Related Art

An endoscope comprising an elongated distal insertion tube to be inserted into an insertion target, the distal insertion tube being incorporated in a curved-shape detection sensor to detect a curved shape (a curved angle and a curved direction) of the distal insertion tube has been known. Such a curved-shape detection sensor is provided with one or more sensing parts to detect a curved shape. The sensor detects the amount of change of detection light at sensing parts by light detector, thereby detecting the curved shape of the distal insertion tube.

For example, Jpn. Pat. Appln. KOKAI Publication No. 2007-44402 discloses an endoscope apparatus comprising a light guide formed of a plurality of optical fibers, a plurality of curvature detection fibers, a filter, and a light receiving element. In the endoscope apparatus, the plurality of curvature detection fibers are arranged on an outer peripheral surface of the light guide put into the insertion tube of the endoscope. The light guide and the curvature detection fibers extend along the insertion tube to the distal end. The filter covers an exit end of the light guide and entrance ends of the curvature detection fibers. Furthermore, a sensing part (an optical loss portion) is provided in each curvature detection fiber in a predetermined position and a predetermined orientation.

In the endoscope apparatus, light emitted from a light source to the entrance end of the light guide is guided from the exit end of the light guide through the filter to the entrance end of each curvature detection fibers. Part of the guided light is lost when passing through the sensing parts in the curvature detection fibers. Light that has passed through the sensing parts without loss is guided to the exit ends of the respective curvature detection fibers. The light receiving element then detects a curved shape of the curvature detection fibers in the sensing part based on the amount of light received from the exit ends of the curvature detection fibers.

### BRIEF SUMMARY OF THE INVENTION

One embodiment of the present invention is an endoscope apparatus comprising an endoscope including a flexible insertion tube; and a curved-shape detection sensor, which includes an optical fiber that transmits detection light and a sensing part provided in at least a part of the optical fiber, and detects a curved shape of the insertion tube based on a change in characteristics of the detection light passed through the sensing part in accordance with a change in the curved shape of the optical fiber when the optical fiber curves, wherein a part of the optical fiber or a part of a guide member through which the optical fiber is passed is held to a component having greater torsion stiffness than any other component constituting the insertion tube.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view for describing a principle of a curved-shape detection sensor.

FIG. 2 is a cross-sectional view taken in a radial direction of a detection light optical fiber.

FIG. 3 is a view showing an overall configuration of an endoscope apparatus including an endoscope on which a curved-shape detection sensor is mounted.

FIG. 4 is a cross-sectional view of a distal insertion tube (free curve portion) of an endoscope apparatus according to a first embodiment, taken in a radial direction.

FIG. 5 is a cross-sectional view of the distal insertion tube of the endoscope apparatus according to the first embodiment, taken in an axial direction.

FIG. 6 is a cross-sectional view of a part of the distal insertion tube, taken in a radial direction along a line B-B in FIG. 5.

FIG. 7 is a cross-sectional view of a distal insertion tube of an endoscope apparatus according to a second embodiment, taken in a radial direction.

FIG. 8 is a cross-sectional view of the distal insertion tube of the endoscope apparatus according to the second embodiment, taken in an axial direction.

FIG. 9 is a cross-sectional view of a distal insertion tube of an endoscope apparatus according to a third embodiment, taken in a radial direction.

FIG. 10 is a cross-sectional view of the distal insertion tube of the endoscope apparatus according to the third embodiment, taken in a radial direction.

### DETAILED DESCRIPTION OF THE INVENTION

### First Embodiment

(Curved-Shape Detection Sensor)

First, a configuration and an operation of a curved-shape detection sensor (hereinafter referred to simply as “sensor”) will be described.

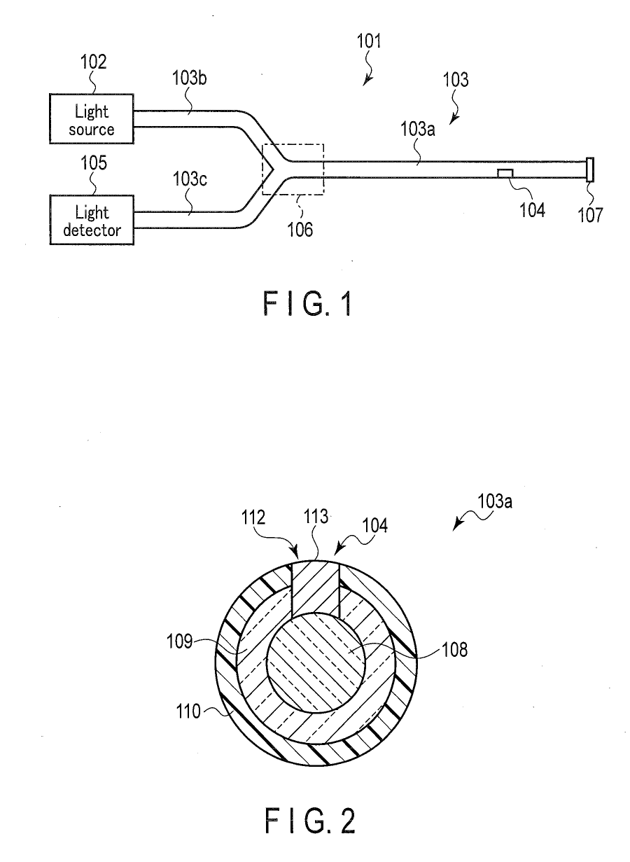

FIG. 1 is a schematic view for describing a principle of the sensor 101. The sensor 101 comprises a light source 102, an optical fiber 103, and a light detector 105. The optical fiber 103 is connected to the light source 102 and the light detector 105. The light source 102 is, for example, an LED light source or a laser light source, which emits detection light having desired wavelength characteristics. The optical fiber 103 transmits the detection light emitted from the light source 102. The light detector 105 detects the detection light guided through the optical fiber 103.

The optical fiber 103 comprises a detection light optical fiber 103a, a light-supplying optical fiber 103b, and a light-receiving optical fiber 103c, which are branched in three ways at a coupler (optical coupler) 106. That is, the optical fiber 103 is formed by connecting two light guide path members, i.e., the light-supplying optical fiber 103b and the light-receiving optical fiber 103c, to one light guide path member, i.e., the detection light optical fiber 103a by the coupler 106. A proximal end of the light-supplying optical fiber 103b is connected to the light source 102. A reflector 107, which reflects the transmitted light, is provided at the distal end of the detection light optical fiber 103a. The reflector 107 is, for example, a mirror. A proximal end of the light-receiving optical fiber 103c is connected to the light detector 105.

The light-supplying optical fiber 103b transmits light emitted from the light source 102 and guides it to the coupler 106. The coupler 106 guides most of the light supplied through the light-supplying optical fiber 103b to the detection light optical fiber 103a, and guides at least a part of the light reflected by the reflector 107 to the light-receiving optical fiber 103c. Furthermore, the light detector 105 receives the light through the light-receiving optical fiber 103c. The light detector 105 photoelectrically converts the received detection light, and outputs an electric signal indicative of an amount of the detection light.

FIG. 2 is a cross-sectional view of the detection light optical fiber 103a, taken in a radial direction. The detection light optical fiber 103a comprises a core 108, a cladding 109 that covers an outer peripheral surface of the core 108, and a coating 110 that covers an outer peripheral surface of the cladding 109. The detection light optical fiber 103a also comprises at least one sensing part 104. The sensing part 104 is provided in only a part of the outer peripheral surface of the detection light optical fiber 103a, and changes characteristics of light passing therethrough in accordance with a change in curved shape of the detection light optical fiber 103a.

The sensing part 104 comprises a light opening 112 which is formed by removing parts of the coating 110 and the cladding 109 to expose the core 108, and an optical characteristic converter 113 formed in the light opening 112. The light opening 112 does not necessarily expose the core 108. The core 108 need not be exposed as long as the light passing through the detection light optical fiber 103a reaches the optical opening 112. The optical characteristic converter 113 converts the characteristics of the light guided through the detection light optical fiber 103a, and is, for example, a guided light loss member (light absorber), a wavelength converter (fluorescent material), or the like. In the following description, the optical characteristic converter is assumed to be a guided light loss member.

In the sensor 101, the light supplied from the light source 102 is guided through the detection light optical fiber 103a, as described above. When the light enters the optical characteristic converter 113 of the sensing part 104, part of the light is absorbed by the optical characteristic converter 113, which causes loss of the guided light. The amount of the loss of the guided light varies in accordance with the amount of curve of the detection light optical fiber 103a.

For example, even if the detection light optical fiber 103a is in a straight state, a certain amount of light is lost in the optical characteristic converter 113 in accordance with the width, length, etc. of the light opening 112. The amount of light lost in the straight state is used as a reference. When the optical characteristic converter 113 is located on an outer side, where the radius of curvature is relatively large, of the detection light optical fiber 103a in its curved state, the amount of loss of the guide light is more than the reference amount of lost light. If the optical characteristic converter 113 is located on an inner side, where the radius of curvature is relatively small, of the curved detection light optical fiber 103a in its curved state, the amount of loss of the guide light is less than the reference amount of lost light.

The change in the amount of loss of the guide light is reflected in the amount of detected light received by the light detector 105, that is, the output signal from the light detector 105. Thus, the curved shape at the position of the sensing part 104 of the sensor 101, that is the position where the optical characteristic converter 113 is provided, can be obtained by the output signal from the light detector 105.

The detection light optical fiber 103a of the sensor 101 is integrally attached to a long flexible curved target to be measured, in the present embodiment, which is a distal insertion tube 11 of an endoscope 10 to be described later, along with the target. The sensor 101 is attached to an appropriate position of the distal insertion tube 11 by positioning a desired detection position of the distal insertion tube 11 to the sensing part 104 of the sensor 101. The detection light optical fiber 103a is curved following a flexible operation of the distal insertion tube 11, and the sensor 101 detects the curved shape of the distal insertion tube 11 as described above.

(Configuration of Endoscope Apparatus)

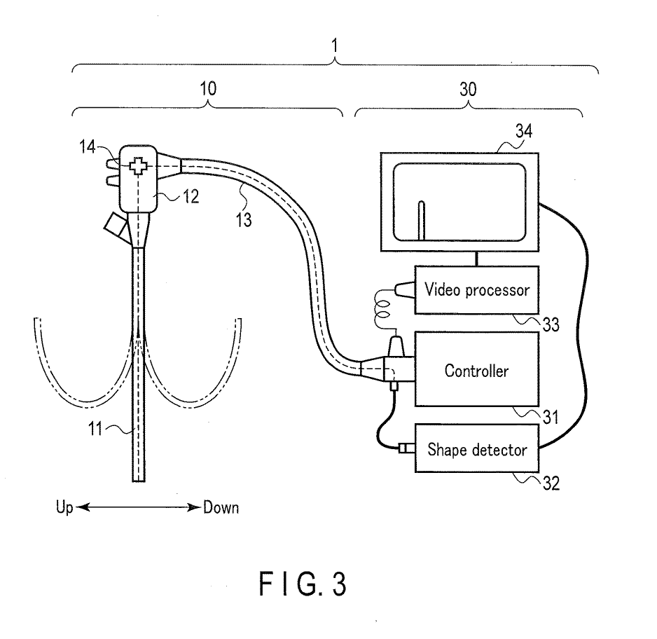

FIG. 3 is a view showing an overall configuration of an endoscope apparatus 1. The endoscope apparatus 1 comprises the endoscope 10 into which at least the detection light optical fiber 103a of the sensor 101 is incorporated and an apparatus main body 30. The apparatus main body 30 comprises a controller 31, a shape detection device 32, a video processor 33, and a monitor 34. The controller 31 controls given functions of the endoscope 10, the shape detection device 32, and the video processor 33 as well as those of peripheral devices connected thereto. Although FIG. 3 does not show the sensor 101, the endoscope apparatus 1 includes the components of the sensor 101 shown in FIG. 1.

The endoscope 10 comprises the flexible distal insertion tube 11 to be inserted into an insertion target, and an operation section 12 provided in a proximal end side of the distal insertion tube 11. A cord section 13 extends from the operation section 12. The endoscope 10 is attachably and detachably connected to the apparatus main body 30 via the cord section 13, and communicates with the apparatus main body 30. The operation section 12 comprises an operation dial 14 with which an operation to curve the distal insertion tube 11 (a curve portion 16 to be described later) in at least two directions (for example, upward and downward) at a desired radius of curvature is input. The cord section 13 contains a first member 25, a second member 26, etc., which are described later.

The endoscope apparatus 1 comprises the sensor 101, and the detection light optical fiber 103a is arranged in the distal insertion tube 11 of the endoscope 10. As described above, when the detection light optical fiber 103a is curved, the sensor 101 detects the curved shape of the distal insertion tube 11 based on a change in characteristics of the detected light (the amount of light in the present embodiment) passed through the sensing part 104 (sensing parts 104b and 104c to be described later) in accordance with a change in the curved shape.

The shape detection device 32 is connected to the light detector 105 of the sensor 101. The shape detection device 32 receives an output signal from the light detector 105 and calculates a curved shape of the distal insertion tube 11 based on the output signal. The calculated curved shape is transmitted from the shape detection device 32 to the monitor 34, and displayed in the monitor 34.

The video processor 33 image-processes an electric signal acquired through the cord section 13 and the controller 31 from an electric signal wiring connected to an image sensor (not shown) at the distal end of the endoscope. The monitor 34 displays an image of an interior of the insertion target processed by the video processor 33.

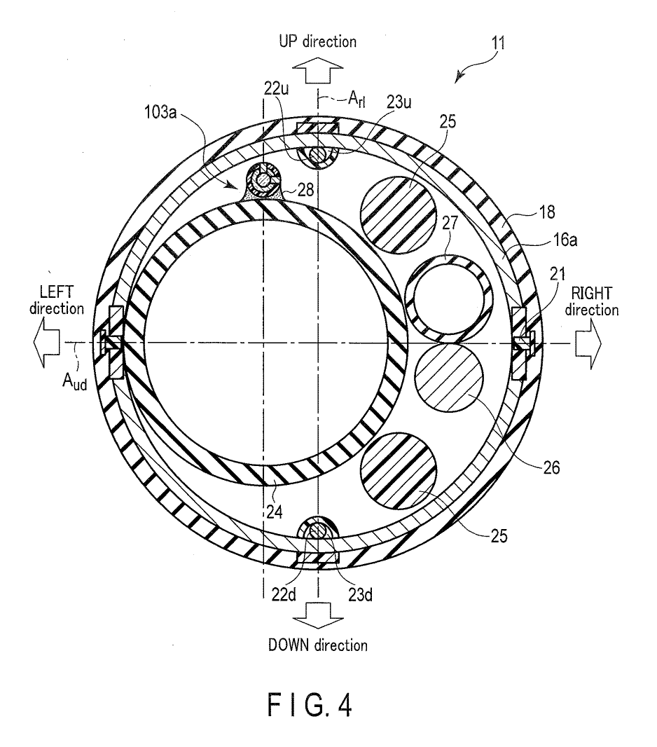

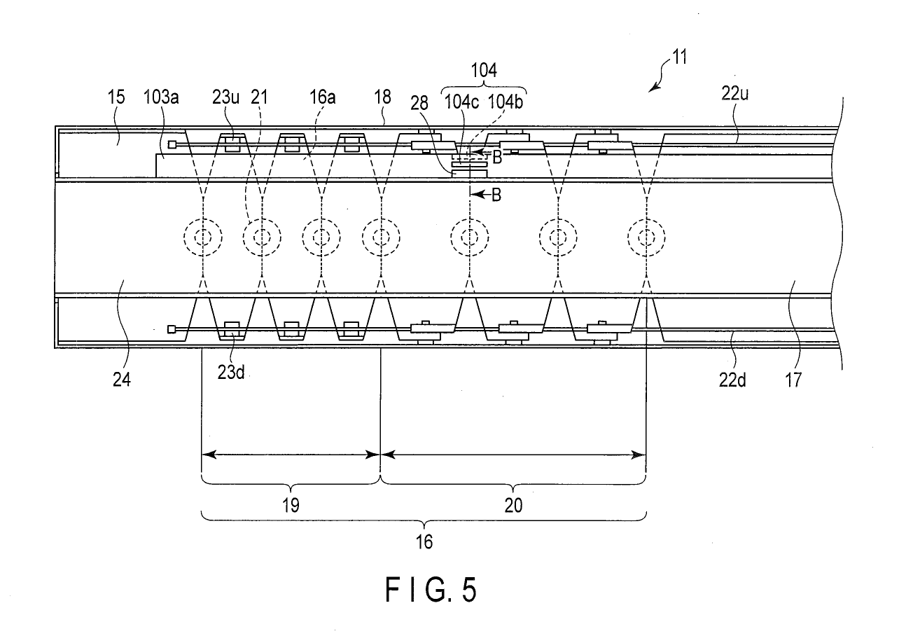

FIG. 4 is a cross-sectional view of the distal insertion tube 11 (a free curve portion 20) of the first embodiment, taken in a radial direction. FIG. 5 is a cross-sectional view of the distal insertion tube 11 in the first embodiment, taken in an axial direction. The distal insertion tube 11 is an elongated cylindrical member on a distal end side of the endoscope. As shown in FIG. 5, the distal insertion tube 11 comprises a rigid distal portion 15, a curve portion 16 including a plurality of pieces 16a having cylindrical shells (cylindrical shell components), and a corrugated tube 17. The pieces 16a are formed of metal, such as stainless steel. The pieces 16a are connected in series in the axial direction of the curve portion 16, while the distal portion 15 is located on a distal end side. Furthermore, the corrugated tube 17 which curves in a free direction is connected to a proximal end side of the curve portion 16 including the pieces 16a. The outer peripheral surfaces of the curve portion 16 (the pieces 16a) and the corrugated tube 17 are covered with a flexible coating 18.

The curve portion 16 is divided into an operation curve portion 19 on the distal end side, which curves in only two directions upward and downward (UP/DOWN, hereinafter referred to as UD) , and a free curve portion 20 on the proximal end side, which curves in four directions upward and downward and rightward and leftward (RIGHT/LEFT, hereinafter referred to as RL) (that can curve 360° in any direction by a combination thereof). Specifically, in the operation curve portion 19, the pieces 16a curve in UD directions with respect to a UD curve axis A_{ud} (see FIG. 4). In the free curve portion 20, the pieces 16a curve in UD directions with respect to the UD curve axis A_{ud} and in RL directions with respect to an RL curve axis Aᵣₗ (see also FIG. 4) perpendicular to the UD curve axis A_{ud}.

In the range of the operation curve portion 19, as shown in FIG. 4, the pieces 16a are connected to one another via rivets 21 on the UD curve axis A_{ud}. Thus, the pieces 16a are connected so as to rotate around the UD curve axis A_{ud}. In the range of the free curve portion 20, the pieces 16a are connected so as to rotate around not only the UD curve axis A_{ud}, but also the RL curve axis Aᵣₗ, which is arranged to be shifted by 90° with respect to a central axis from the UD curve axis A_{ud}.

As shown in FIG. 5, distal ends of an operation wire 22u for curving upward and an operation wire 22d for curving downward are fixed to the distal portion 15 of the distal insertion tube 11. The operation wires 22u and 22d are respectively inserted through recesses 23u and 23d of the pieces 16a in the curve portion 16. Proximal ends of the operation wires are connected to the operation dial 14 of the operation section 12. With this structure, the curve portion 16 of the distal end insertion tube 11 curves upward when the operator rotates the operation dial 14 and the operation wire 22u is pulled, and curves downward when the operation wire 22d is pulled.

The UD curve axis A_{ud} and the RL curve axis Aᵣₗ are rotation axes defined by the rivets 21, and present at each of the rivets 21 connecting the pieces 16a. The rivets 21 are parallel to one another. Also, when the distal insertion tube 11 as a whole is viewed, an imaginary central axis of curving is parallel to the rivets 21. Alternatively, without using the rivets 21 that define a curving direction, the pieces 16a may have a structure which defines the curving direction by means of, for example, a groove machined in a pipe material. This structure also has an imaginary central axis of curving. In either of the structures described above, the imaginary central axis of curving is nearly perpendicular to the operation wires 22u and 22d.

Inside the distal insertion tube 11, as shown in FIG. 4, a channel tube 24, at least one first member 25, at least one second member 26 and at least one third member 27 extend in a longitudinal direction. The first members 25, the second member 26 and the third member 27 are, respectively, one selected from a light guide, an image guide, a wire for an electric signal from an image sensor, a wire for power supply, an air supply tube, a water supply tube, an operation wire, etc. The channel tube 24 is a cylindrical tube which allows passage of a treatment tool, such as an ultrasonic probe or forceps. For example, the light guide is connected to an illumination optical system (not shown) contained in the distal portion 15 at a distal end thereof, and to a light source (not shown) through the cord section 13 at a proximal end thereof. For example, the wire for an electric signal is connected at a distal end thereof to an image sensor (not shown) contained in the distal portion 15, and at a proximal end thereof to the controller 31 through the cord section 13.

The detection light optical fiber 103a of the sensor 101 is curvably joined together with the channel tube 24 and held on an outer peripheral surface of the channel tube 24 by adhesive 28, as shown in FIG. 4 and FIG. 5. An adhesion position in the axial direction in the detection light optical fiber 103a with respect to the channel tube 24 is one position just under the sensing part 104 (sensing parts 104b and 104c to be described later) of the detection light optical fiber 103a in the radial direction, as shown in FIG. 5. The adhesion position may be in the vicinity of the distal end of the detection light optical fiber 103a, but it is preferable that only one adhesion position is applied to reduce the number of places where bending stress caused by the adhesion occurs. If the vicinity of the sensing part 104 is adhered, it is preferable that the adhesive has elasticity (for example, a silicone adhesive). The joining is not limited to adhesion but may be fusion.

The component that holds the detection light optical fiber 103a is not limited to the channel tube 24, but may be the operation wire 22u or 22d, the first member 25, the second member 26, the third member 27, etc., which curves inside the distal insertion tube 11. Here, since the channel tube 24 is the largest in diameter of all internal components of the distal end insertion tube 11, it has greater torsional stiffness than that of any other internal components. If the internal component to which the detection light optical fiber 103a adheres is twisted, the position of the sensing part 104 may be displaced and it causes less accurateness of detecting the curved shape. Therefore, it is desirable that the detection light optical fiber 103a be attached to an internal component that has greater torsional stiffness. For the reasons stated above, in the present embodiment, the channel tube 24 that has the greatest torsional stiffness of all components constituting the distal insertion tube 11 is used as a sensor holding member, and a part of the detection light optical fiber 103a is held on the channel tube 24.

From the viewpoint as described above, it is preferable that the channel tube 24 has an outer diameter larger than ½ of the inner diameter of the pieces 16a, and torsional stiffness of the channel tube 24 is greater than that of the detection light optical fiber 103a, for example, the channel tube 24 has a strength of twice or more of the detection light optical fiber 103a with regard to the torsional stiffness.

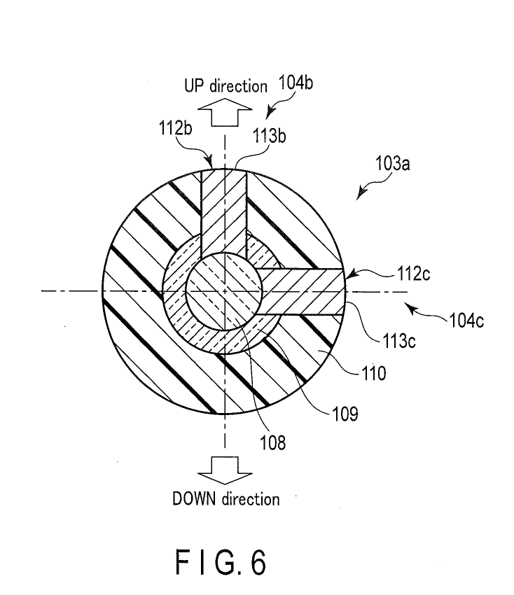

FIG. 6 is a cross-sectional view taken in a radial direction along a line B-B in FIG. 5, and including a sensing part 104b (a light opening 112b and a optical characteristic converter 113b) and a sensing part 104c (a light opening 112c and a optical characteristic converter 113c) in the free curve portion 20. Since the free curve portion 20 is curved in the UD directions and the RI directions, the free curve portion 20 has the sensing part 104b in a direction corresponding to the UD directions, that is, at a position perpendicular to the UD curve axis A_{ud}, and the sensing part 104c in a direction corresponding to the RL directions, that is, at a position perpendicular to the RL curve axis Aᵣₗ. Thus, the sensing parts 104b and 104c are provided in positions perpendicular to each other, corresponding to the UD directions and the RL directions. The free curve portion 20 of the curve portion 16 curves in the UD and RL directions. Therefore, in order for the detection light optical fiber 103a to detect a curved shape of the distal insertion tube 11 in the range of the free curve portion 20, the two sensing parts 104b and 104c perpendicular to each other as shown in FIG. 6, are arranged in the range of the free curve portion 20. Even if the two sensing parts 104b and 104c are provided in directions perpendicular to each other, as described above, a change in the amount of light guided through the optical fiber 104a for detection light and passed through the sensing parts 104b and 104c is detected by the light detector 105. Based on the detection, the shape detection device 32 calculates a curved shape of the distal insertion tube 11.

The light openings 112b and 112c constituting the sensing parts 104b and 104c are filled with the optical characteristic converters 113b and 113c which absorb light having wavelengths different from each other. The optical characteristic converters 113b and 113c absorb an amount of light of specific different wavelengths (wavelength bands) guided through the detection light optical fiber 103a. Because of the different optical characteristic converters 113b and 113c provided in the light openings 112b and 112c, the light detector 105 can distinguishingly detect a change in the amount of light resulting from curving in the UD directions and a change in the amount of light resulting from curving in the RL directions in the free curve portion 20.

A curve axis in the operation curve portion 19 operable by the operation wires 22u and 22d, that is, a curve axis in a direction curved by operating the operation wires 22u and 22d, is defined as a primary curve axis. In the present embodiment, the primary curve axis is the UD curve axis A_{ud}. For example, if there are a plurality of curve axes in the operation curve portion 19, the curve axis of the greatest curve angle is the primary curve axis.

In the present embodiment, the pieces 16a, each being rotatable around the rivets 21 as a central axis, are connected in series, so that it brings the distal insertion tube 11 of the endoscope being curvable. However, the embodiment may have a structure to make the distal insertion tube 11 curvable by deforming a pipe member machined in a manner having slits. In this case, a member between adjacent slits of the pipe member, which are parallel to each other, serves a function corresponding to a piece 16a. Furthermore, an imaginary axis perpendicular to a central axis of the pipe member and extending from an opening of a slit at an intersection of an imaginary center line of the slit and the central axis of the pipe member serves a function corresponding to the rivets 21.

(Advantages)

When the distal insertion tube 11 is curved by the operator's operating the operation wires 22u and 22d with the operation dial 14 or by receiving external force due to, for example, contact of the distal insertion tube 11 with the insertion target, the detection light optical fiber 103a inside the distal insertion tube 11 is also curved following the curve of the distal insertion tube 11. Here, even if another internal component constituting the distal insertion tube 11 (for example, the first member 25, the second member 26, or the third member 27) is brought into contact with the channel tube 24 and presses the channel tube 24, it is unlikely that the channel tube 24 twists because the outer diameter of the channel tube 24 is larger (thicker) than that of the other component and torsional stiffness thereof is greater than that of the other component. Therefore, it is also unlikely that detection light optical fiber 103a held on the channel tube 24 twists.

According to the present embodiment, the detection light optical fiber 103a is attached to the channel tube 24 having greater torsional stiffness than any other internal component constituting the distal insertion tube 11 and therefore is unlikely to get twisted. Thus, the directions of the sensing parts 104b and 104c do not easily change due to an influence of a twist in the detection light optical fiber 103a. Therefore, the curved shape of the distal insertion tube 11 can be accurately detected without lowering the detection accuracy of the curved shape (a radius of curvature and a direction) by the sensor 101.

Moreover, according to the present embodiment, detecting directions of the light openings 112b and 112c are set in accordance with the UD curve axis A_{ud} and the RL curve axis Aᵣₗ, that is, are set perpendicular to those curve axes. Therefore, the curved shape in the detecting directions can be detected with high sensitivity.

Thus, according to the present embodiment, it is possible to provide an endoscope apparatus that enables accurate detection of a curved shape of the distal insertion tube 11.

### Second Embodiment

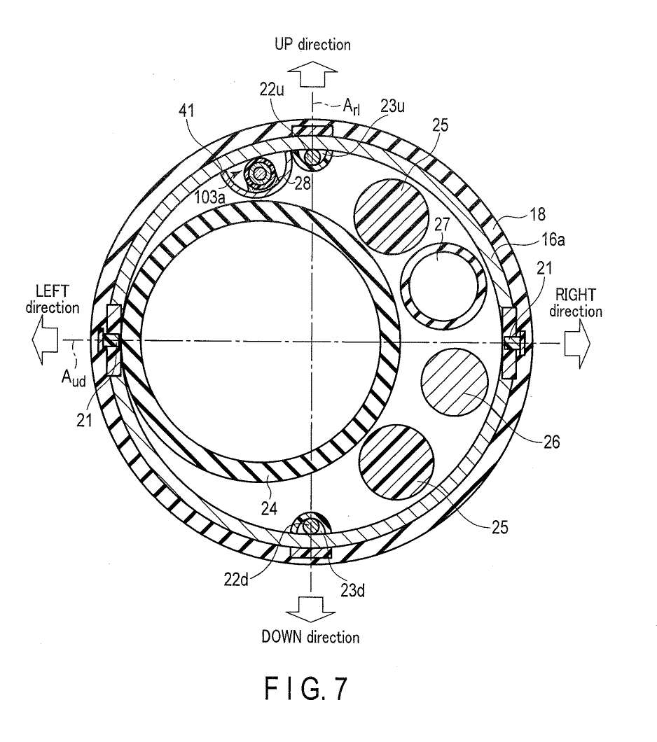

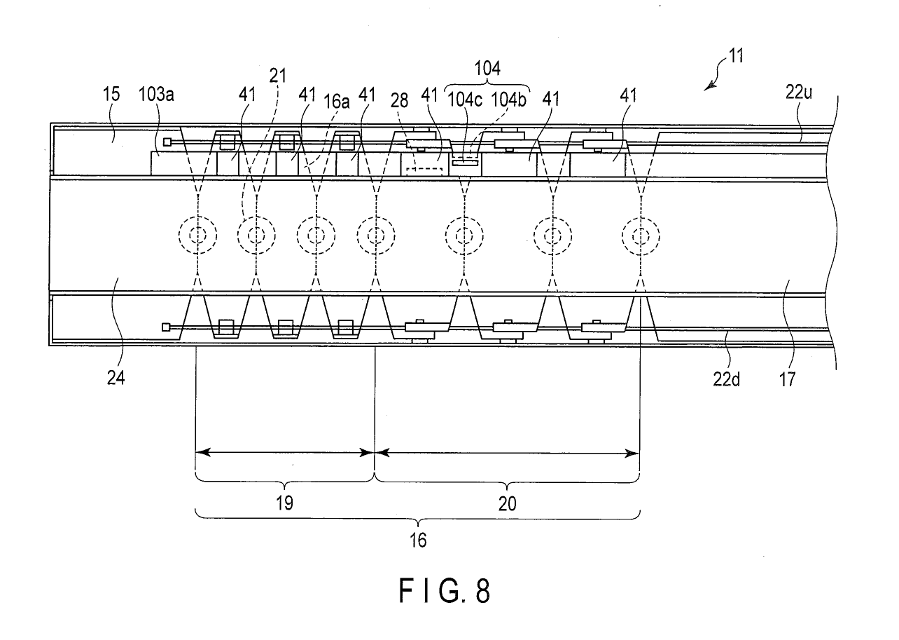

The second embodiment of the present invention will be described with reference to FIG. 7 and FIG. 8. In the following, the same reference numerals as used in the first embodiment will be used for the same parts, and detailed descriptions thereof will be omitted, and only matters different from the first embodiment will be described.

(Configuration)

In the present embodiment, a plurality of sensor bulges 41 as guide members for a detection light optical fiber 103a are respectively provided on pieces 16a in a curve portion 16 inside a distal insertion tube 11. Each of the sensor bulges 41 is an almost semicircular member bulging radially inward from an inner surface of the piece 16a. The sensor bulge 41 has an inner diameter greater than the outer diameter of the detection light optical fiber 103a. The detection light optical fiber 103a is inserted through the sensor bulge 41 and held on the piece 16a via the sensor bulge 41.

The detection light optical fiber 103a is curvably connected to the piece 16a with adhesive applied between an outer surface of the detection light optical fiber 103a and an inner surface of only one of the sensor bulges 41, that is, in only one of the pieces 16a. The piece 16a to which the detection light optical fiber 103a adheres is one that is located in the vicinity of the sensing part 104 of the optical fiber 103a to maintain the position and facing condition of the sensing part 104 (sensing parts 104b and 104c). The detection light optical fiber 103a is slidable in the axial direction relative to sensor bulges other than the sensor bulge to which it adheres.

The detection light optical fiber 103a may be held to the distal insertion tube 11 by adhesion of the distal end thereof to the distal portion 15. In this case, the detection light optical fiber 103a can be held so as to be axially slidable relative to the sensor bulges 41 of all pieces 16a.

(Advantages)

The diameter of the piece 16a is the largest (thickest) of all components constituting the distal insertion tube 11 (that is, larger than the diameter of any internal component (the channel tube 24 etc.) constituting the distal insertion tube 11). The pieces 16a are made of metal, such as stainless steel, which is resistant to twist. Stiffness of the connected pieces 16a as a whole is slightly reduced by rattling etc. of rivets 21, but it has little influence of the rattling. When the distal insertion tube 11 is curved, if the adjacent pieces 16a are brought into contact with each other, the pieces 16a cannot be twisted any more. Therefore, the overall stiffness of the connected pieces 16a that is sufficient for practice is ensured, and results in lower twistability.

The sensor bulge 41 functions as a guide member which guides sliding in the axial direction of the detection light optical fiber 103a to eliminate a difference in length between an inner side and an outer side of a curve of the detection light optical fiber 103a. The guide makes the detection light optical fiber 103a less twistable. In addition, it reduces the risk that the optical fiber 103a may be in contact and interfere with another internal component.

Furthermore, since the detection light optical fiber 103a is inserted through the sensor bulge 41, the detection light optical fiber 103a is protected by the sensor bulge 41. Therefore, the detection light optical fiber 103a does not easily interfere with another internal component contained in the distal insertion tube 11 (for example, the first member 25, the second member 26 or the third member 27). Accordingly, it becomes difficult for twisting of the detection light optical fiber 103a to occur.

The piece 16a is made of metal that is resistant to twisting, as described above, and has high rigidity. Therefore, if the detection light optical fiber 103a adheres to the piece 16a within the length in the axial direction of the pieces 16a, it increases the adhesion strength of the detection light optical fiber 103a to the distal insertion tube 11 and improves the reliability of the accuracy of detecting a curved state.

As described above, the present embodiment can also provide an endoscope apparatus that enables more accurate detection of a curved shape of the distal insertion tube 11.

### Third Embodiment

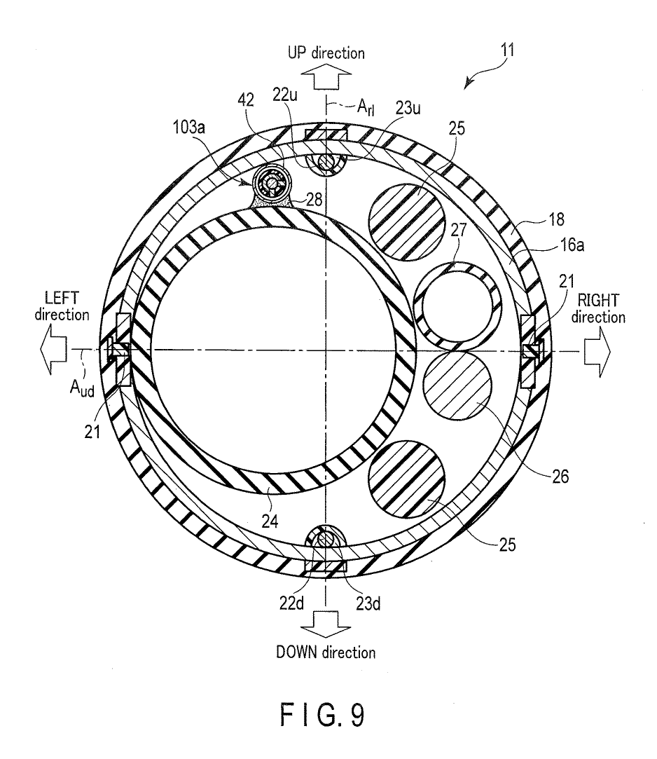

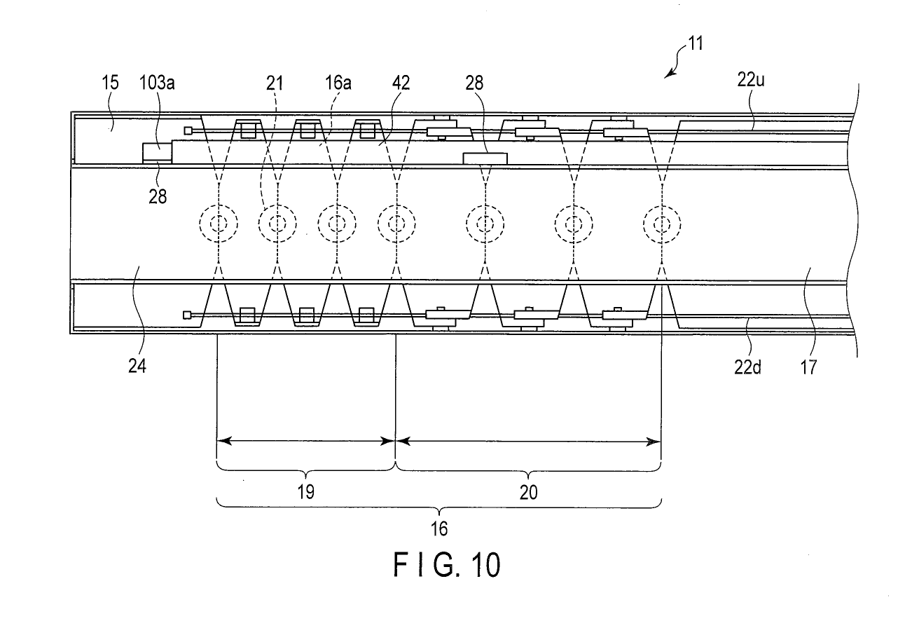

The third embodiment of the present invention will be described with reference to FIG. 9 and FIG. 10. In the following, the same reference numerals as used in the second embodiment will be used for the same parts, and detailed descriptions thereof will be omitted and only matters different from the second embodiment will be described.

(Configuration)

In the present embodiment, a cylindrical sensor coil 42 as a guide member of the detection light optical fiber 103a is arranged on an outer peripheral surface of the detection light optical fiber 103. In other words, the detection light optical fiber 103a is inserted through the sensor coil 42 so as to be slidable in an axial direction. The sensor coil 42 has an inner diameter larger than the outer diameter of the detection light optical fiber 103a.

The length of the sensor coil 42 is somewhat shorter than that of a distal insertion tube 11 (or a channel tube 24). The sensor coil 42 is held along the channel tube 24, starting from a position slightly shifted from the distal end of the channel tube 24 toward the proximal end. In other words, the distal end of the detection light optical fiber 103a slightly projects from the distal end of the sensor coil 42 in the axial direction. The projected part of the distal end portion of the detection light optical fiber 103a is held to the channel tube 24 by adhesion (or fusion).

Furthermore, the sensor coil 42 is held to the channel tube 24 by adhesion (or fusion) in only one place (one point) in vicinity of the sensing part 104 of the detection light optical fiber 103a. The point to which the sensor coil adheres is one that is located in the vicinity of the sensing part 104 of the optical fiber 103a to maintain the position and facing condition of the sensing part 104. The adhering position may be any other position; for example, the sensor coil 42 may be held by adhesion at any other position, such as the distal end thereof.

The sensor coil 42 is, for example, a coil spring, and has elasticity equal to or greater than that of the channel tube 24. The sensor coil 42 may be caused to adhere to the channel tube 24 by, for example, elastic adhesive. The sensor coil 42 may be caused to adhere in the overall length or at intervals at points, that is, a plurality of adhering points may be interspersed. The sensor coil 42 may be formed of a material that curves following the curve of the distal insertion tube 11, for example, a fluororesin tube.

The length of the sensor coil 42 in the axial direction may be smaller than that of the channel tube 24, and may cover the detection light optical fiber 103a in a desired range (for example, the operation curve portion 19 or the free curve portion 20).

The sensor coil 42 may be held to one or more of the pieces 16a in the distal insertion tube 11. In this case, the sensor coil 42 may adhere to at least one desired piece 16a of the pieces 16a; however, it may adhere to two or more pieces 16a, including all pieces 16a. If the sensor coil 42 adheres to a piece 16a, the adhesive need not be an elastic adhesive, but may be a hard adhesive, such as an epoxy adhesive.

(Advantages)

In the present embodiment, the sensor coil 42 is held by adhesion or the like in one place (one point) of the channel tube 24 or the piece 16a. Therefore, the sensor coil 42 does not receive bending stress other than an adhered portion, even if the distal insertion tube 11 is curved.

Furthermore, when the distal insertion tube 11 curves, the components also similarly curve. For example, when the distal insertion tube 11 curves in the UP direction, the sensor coil 42 curves inward and accordingly receives compression bending stress in the adhered portion. When the distal insertion tube 11 curves in the DOWN direction, the sensor coil curves outward and accordingly receives tensile bending stress in the adhered portion. In either case, the sensor coil 42 is extensible and compressible as well as the channel tube 24.

The detection light optical fiber 103a itself is flexible, but is not extensible or compressible. However, since the sensor coil 42 is held to the channel tube 24 or the piece 16a at only one point, the detection light optical fiber 103a slides in the axial direction within the sensor coil 42 when the distal insertion tube 11 curves. Thus, even when the distal insertion tube 11 is bent, bending stress does not occur in the detection light optical fiber 103a.

Moreover, since the optical fiber 103a is encircled by the sensor coil 42, it does not easily interfere with another internal component (for example, the first member 25, the second member 26 or the third member 27) contained in the distal insertion tube 11. Therefore, it becomes difficult for twisting of the detection light optical fiber 103a to occur. Also, it is unlikely that the detection light optical fiber 103a buckles.

Thus, the present embodiment can provide an endoscope apparatus that enables more accurate detection of a curved shape of the distal insertion tube 11 than the first and second embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope apparatus comprising:
an endoscope including a flexible insertion tube; and
a curved-shape detection sensor, which includes an optical fiber that transmits detection light and a sensing part provided in at least a part of the optical fiber, and detects a curved shape of the insertion tube based on a change in characteristics of the detection light passed through the sensing part in accordance with a change in the curved shape of the optical fiber when the optical fiber curves,
wherein a part of the optical fiber or a part of a guide member through which the optical fiber is passed is held to a component having greater torsion stiffness than any other component constituting the insertion tube.

2. The endoscope apparatus according to claim 1, wherein the component having greater torsion stiffness is greater in diameter than any other component constituting the insertion tube.

3. The endoscope apparatus according to claim 2, wherein the component holding the part of the optical fiber or the part of the guide member is a channel tube.

4. The endoscope apparatus according to claim 2, wherein the component holding the part of the optical fiber or the part of the guide member comprises a plurality of cylindrical shell components.

5. The endoscope apparatus according to claim 4, wherein the part of the optical fiber or the part of the guide member is fixed to only one of the cylindrical shell components, and axially slidable with respect to other cylindrical shell components.

6. The endoscope apparatus according to claim 5, wherein the one of the cylindrical shell components is located in vicinity of the sensing part.

7. The endoscope apparatus according to claim 3, wherein the guide member is fixed to the channel tube at a point, and the optical fiber is axially slidable within the guide member.

8. The endoscope apparatus according to claim 7, wherein the point at which the guide member is fixed is located in vicinity of the sensing part.

9. The endoscope apparatus according to claim 1, wherein the component having greater torsion stiffness has torsion stiffness twice or more of that of the optical fiber.

10. The endoscope apparatus according to claim 1, wherein the component having greater torsion stiffness is one selected from a cylindrical shell component, a channel tube, a light guide, an image guide, a wire for an electric signal, a wire for power supply, an air supply tube, a water supply tube and an operation wire.
